(19) United States
(12) Patent Application Publication
(10) Pub. No.: US 2024/0350225 A1
(43) Pub. Date: Oct. 24, 2024

(54) SURGICAL TABLE WITH INSET HIGH-INTENSITY LIGHT SOURCE

(71) Applicant: Richard Bourque, San Jose, CA (US)

(72) Inventor: Richard Bourque, San Jose, CA (US)

(21) Appl. No.: 18/135,959

(22) Filed: Apr. 18, 2023

(51) Int. Cl. A61B 90/35; A61G 13/10

(52) U.S. Cl. CPC A61B 90/35; A61G 13/10; A61B 2090/309

(57) ABSTRACT

Embodiments of the present invention provide a table with an inset, high-intensity light source that can illuminate a patient for improved visibility of the patient's anatomy during surgery and medical examinations, for example. The light source can be disposed inside of the table and illuminates the patient's body from underneath the body as the patient lays on the table. The light source can be integral to the structure of the table, or the light source can be integrated into a cushion, cover, or other aspect of the table. According to some embodiments, the inset lighting is removeable, or is integral to a removeable component of the table, such as a removeable cushion, attachment, etc.

### FIELD

Embodiments of the present invention generally relate to the field of medical devices and equipment. More specifically, embodiments relate to lighting equipment used for medical procedures and diagnosis.

### BACKGROUND

In the field of health and medicine, it is important to provide sufficient lighting during medical examinations, surgery, and other similar procedures. High-quality lighting is especially important for accurately and consistently analyzing the internal anatomy of the patient during surgery. Having sufficient illumination is critical for optimizing patient safety and staff comfort, with light emitting diode (LED) surgical lights becoming the standard in most hospitals and medical offices. Poor lighting during a surgical procedure can lead to eye fatigue and may put the patient's safety at risk.

Traditionally, patients in a medical environment are illuminated by an external light source, such as overhead lighting or adjustable lamps, lighting rigs, etc. These types of light sources may be useful for illuminating the surrounding environment but often do not provide enough light that actually reaches the patient's body. For example, external lights can be blocked by people and equipment, which produces shadows on the patient. By illuminating the patient more directly, it is possible to see the patient's body in greater detail, including parts of the anatomy that are below the surface of the skin.

FIG. 1 depicts an exemplary conventional surgical table 100 where illumination is generally from above. During a surgical procedure or medical examination, external lighting is required to make certain areas of the patient's body visible. Moreover, there is no practical way using a conventional surgical table to illuminate the patient's body from underneath the patient, for example.

Accordingly, a need exists in the field of medical equipment and lighting systems for a device that can more directly illuminate a patient's body and improve visibility during medical examination and surgical procedures.

### SUMMARY

Embodiments of the present invention provide a medical equipment table with an inset, high-intensity light source that can illuminate a patient for improved visibility of the patient's anatomy during surgery and medical examinations, for example. The light source is disposed inside of the table and illuminates the patient's body from underneath with an intensity and in a way that is visible from the other side of the body. The light source can be integral to the structure of the table, or the light source can be integrated into a cushion, cover, or other aspect of the table. According to some embodiments, the inset lighting is removeable, or is integral to a removeable component of the table, such as a cushion, attachment, etc.

According to one embodiment of the present invention, a medical equipment structure for illuminating a living body is disclosed. The structure includes a flat supportive element, and a light source disposed within the flat supportive element. The flat supportive element accommodates a living body, and the light source is operable to produce light that illuminates the living body so that an internal anatomy of the body is visible from outside the living body.

According to some embodiments, the flat supportive element includes a surgical table.

According to some embodiments, the flat supportive element includes an examination table.

According to some embodiments, the medical equipment structure includes padding that encloses the flat supportive element, and the light source is partially enclosed by the padding.

According to some embodiments, the medical equipment structure includes a control element coupled to the light source, and the control element is operable to adjust a brightness, intensity, color, or temperature of the light produced by the light source.

According to some embodiments, the medical equipment structure includes including a base that supports the flat supportive element, and the flat supportive element is removably coupled to the base.

According to some embodiments, the light source includes an LED array.

According to another embodiment, a surgical table having an integrated light source is disclosed. The surgical table includes a tabletop including an integrated light source, and a base that supports the tabletop. The tabletop accommodations and supports a body of a patient, and the integrated light source is operable to produce light that illuminates the body of the patient from below when the patient is disposed on the tabletop and directly above the integrated light source.

According to some embodiments, the tabletop includes padding, and the integrated light source is partially enclosed by the padding.

According to some embodiments, the integrated light source includes an LED.

According to some embodiments, the integrated light source includes an LED array.

According to some embodiments, the surgical table includes a control element coupled to the integrated light source, and the control element is operable to adjust a brightness, intensity, color, or temperature of the light produced by the integrated light source.

According to some embodiments, the integrated light source includes different types of light sources, and the control element is further operable to selectively activate the different types of light sources.

According to some embodiments, the integrated light source is operable to illuminate an internal anatomy of the body of the patient so that the internal anatomy is visible from outside of the body of the patient.

According to a different embodiment, a surgical table having a removeable a light source is disclosed. The surgical table includes a tabletop, a removable component including a light source, and a base that supports the tabletop and the removeable component. The removeable component is removably coupled to at least one of the base and the tabletop, and the tabletop accommodations and supports a body of a patient. The light source illuminates the body of the patient from below when the patient is disposed on the tabletop and directly above the light source.

According to some embodiments, the removeable component further includes padding, and the light source is partially enclosed by the padding.

According to some embodiments, the light source includes an LED.

According to some embodiments, the light source includes an LED array.

According to some embodiments, the surgical table includes a control element coupled to the light source, and the control element is operable to adjust a brightness, intensity, color, or temperature of the light produced by the light source.

According to some embodiments, the light source includes different types of light sources, and the control element is further operable to selectively activate the different types of light sources.

According to some embodiments, the light source is operable to illuminate an internal anatomy of the body of the patient so that images representing the internal anatomy of the patient are visible from outside of the body of the patient.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 depicts an exemplary conventional surgical table that is designed for illumination using a light source mounted above.

FIG. 2 depicts an exemplary surgical table with inset high-intensity lighting, such as an LED array, according to embodiments of the present invention.

FIG. 3A is a block diagram depicting exemplary medical equipment with inset high-intensity lighting according to embodiments of the present invention.

FIG. 3B is a block diagram depicting exemplary medical equipment structure with inset high-intensity lighting from a top-down view according to embodiments of the present invention.

FIG. 4 is a diagram depicting an exemplary inset high-intensity light source disposed in a removeable component of medical equipment structure, such as a surgical table or examination table, according to embodiments of the present invention.

FIG. 5 is a diagram depicting an exemplary inset high-intensity light source disposed in a removeable component attached to medical equipment structure according to embodiments of the present invention.

FIG. 6 is a diagram of an exemplary high-intensity light source that can be inset within medical equipment structure to improve the visibility of the practitioner and illuminate aspects of a patient's anatomy (e.g., organs, vasculature, soft-tissue, etc.) by directly illuminating the patient from below according to embodiments of the present invention.

FIG. 7 is a schematic diagram of exemplary medical equipment structure with an inset high-intensity light source for directly illuminating a patient's body from below according to embodiments of the present invention.

FIG. 8 is a schematic diagram of exemplary medical equipment structure with inset high-intensity light sources light sources for directly illuminating a patient's body from below and a cooling system that cools the light sources according to embodiments of the present invention.

FIG. 9 is a diagram of an exemplary cover or shroud for use with a medical equipment structure having inset lighting according to embodiments of the present invention.

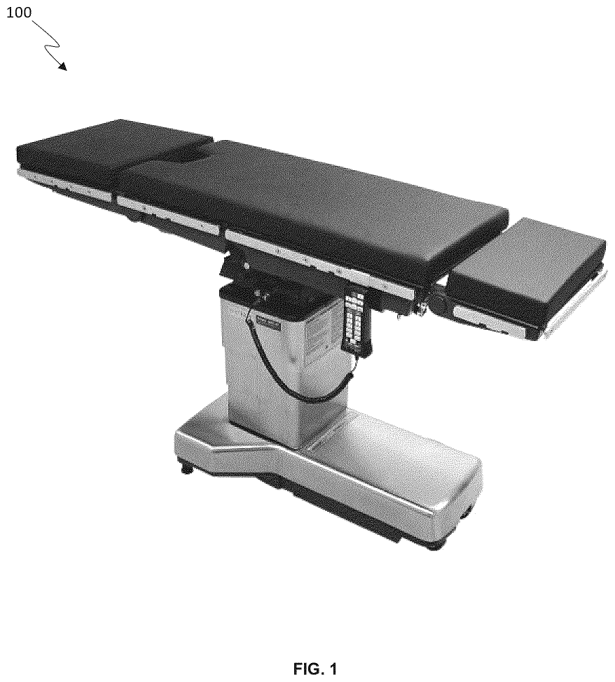

### DETAILED DESCRIPTION:

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart of the figure herein, and in a sequence other than that depicted and described herein.

Some portions of the detailed description are presented in terms of procedures, steps, logic blocks, processing, and other symbolic representations of operations on data bits that can be performed on computer memory. These descriptions and representations are the means used by those skilled in the data processing arts to most effectively convey the substance of their work to others skilled in the art. A procedure, computer-executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout, discussions utilizing terms such as “accessing,” “writing,” “including,” “storing,” “transmitting,” “traversing,” “associating,” “identifying,” “updating,” “determining,” “selecting,” “animating,” “displaying,” “lighting” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

### Surgical Table with Inset High-Intensity Light Source for Illuminating Patient from Under Side

Embodiments of the present invention provide a table with an inset, high-intensity light source that can illuminate a patient for improved visibility of the patient's anatomy during surgery and medical examinations, for example. The light source can be disposed inside of the table and illuminates the patient's body from underneath the body as the patient lays on the table. The light source can be integral to the structure of the table, or the light source can be integrated into a cushion, cover, or other aspect of the table. According to some embodiments, the inset lighting is removeable, or is integral to a removeable component of the table, such as a removeable cushion, attachment, etc. In one example, inset lighting is included in extensions that support the arms/legs or head of the patient, and the extensions can be removeable and replaceable.

Depending on the intensity of the light produced by the inset light source, images of different aspects of the patient's body can be made visible, such as organs, veins, tumors, bones, ligaments, etc. The ability of the medical practitioner to diagnose the patient and perform surgery and other procedures is improved due to the increased visibility and real-time knowledge of the patient's body, including the position, geometry, and function of organs and other biological systems. Advantageously, the visibility provided by the insert high-intensity lighting can obviate the need for separate imaging, such as MRIs, x-rays, etc., which are often expensive and time consuming.

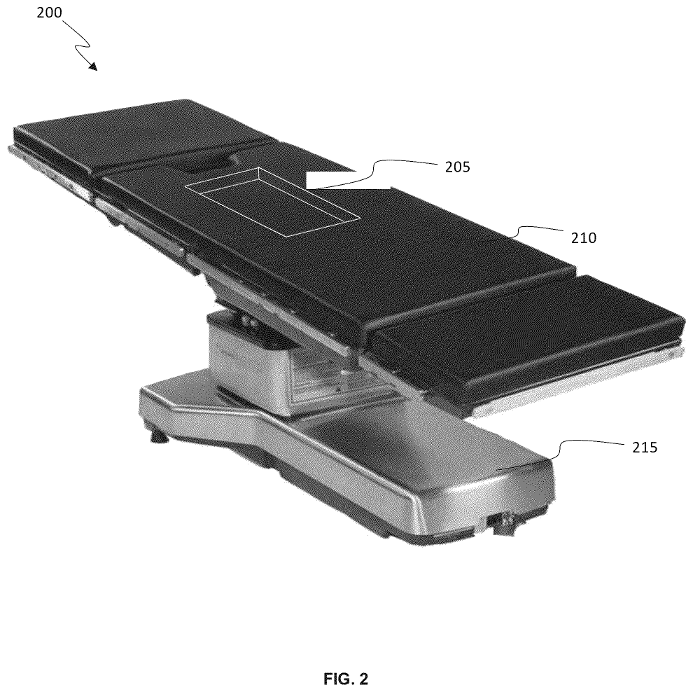

FIG. 2 depicts an exemplary surgical table with inset high-intensity lighting according to embodiments of the present invention. In the example of FIG. 2, inset light source 205 is disposed within table 200 to illuminate a patient using high-intensity lighting. Light source 205 can be a single light source, such as a single LED, or multiple light sources, such as an LED array, or a combination of LEDs and other light sources, such as high-intensity discharge (HID) lights, florescent light, etc. Light source 205 is sufficiently intense to directly illuminate a patient's body from below, and the illumination allows a medical practitioner to observe aspects of the patient's anatomy that would not be visible under typical lighting conditions using an external lighting rig, overhead lighting, etc. Light source 205 can be fixed inside of the table, or can be removeable according to embodiments. Table 200 can include a tabletop 210 that is supported by a base 215, and the base 215 can include legs, wheels, etc. Table 200 is typically made from sterile, leak proof components.

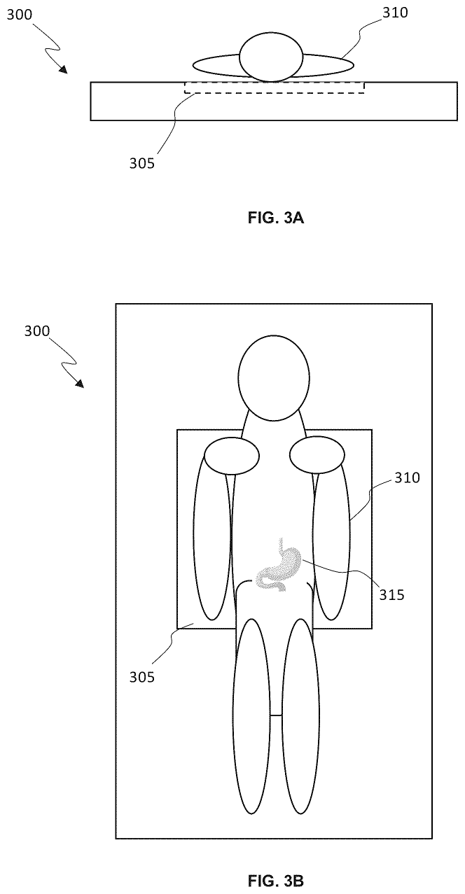

FIG. 3A is a block diagram depicting exemplary medical equipment structure 300 with inset high-intensity lighting according to embodiments of the present invention. FIG. 3B is a block diagram depicting exemplary medical equipment structure 300 with inset high-intensity lighting from a top-down view according to embodiments of the present invention. Medical equipment structure 300 can be a simple furniture structure, a surgical table, an examination table, an adjustable chair, or the like. In a basic embodiment, medical equipment structure 300 is a flat supportive structure that receives or encloses a light source 705, or a housing or ballast thereof, and allows light from light source 705 to reach the patient 310.

As depicted in FIG. 3A, patient 310 lays flat on the surface of medical equipment structure 300 on top of light source 305. The light-source 305 can be covered by a transparent lens or material made from glass (e.g., tempered glass), plastic, acrylic and the like to support the patient's body and to provide a barrier between the patient's body 310 and light source 305. Some embodiments use a thin transparent gel disposed on the cover to further insulate patient 310 from light source 305 and improve the comfort of the patient. A similar result can be achieved using open-cell foam padding, according to embodiments. Patient 310 can lay on medical equipment structure 300 while oriented flat on their back, face-up, on their side, etc., to illuminate different aspects of the anatomy.

As the high-intensity lighting shines through the patient's body from below, shadows are advantageously eliminated and images of aspects of the patient's anatomy 315 are revealed and made easily visible during examination, surgery, etc. Accordingly, patient 310 can often be treated or examined without needing separate imaging or diagnostic procedures, which reduces cost of treatment and improves the efficiency of treatment and patient care. Moreover, according to some embodiments, light-source 305 is controllable and configurable via a control system that can adjust the intensity/brightness, color, and temperature of the light as desired to achieve a desired result. For example, some aspects of body 310 may require more brightness to be illuminated sufficiently, or may be illuminated better by a different quality of light (e.g., a different color or temperature). Moreover, some aspects of a patient's body may respond differently to different colors of light, and the color of light sources can be adjusted to better illuminate a target region or organ. The light color can be selected and used in combination with filtered eyeglasses that selectively block certain wavelengths/colors to enhance visibility during a medical procedure. Furthermore, some embodiments of the present invention provide different types of light sources (LED, HID, fluorescent, etc.), and the different light sources can be selectively activated to achieve the desired effect, for example, to illuminate tissues of different densities. Typically, an LED or LED array is used to illuminate patient 310 due to the high brightness and low heat dissipation thereof, although any relatively compact high-intensity light source can be used.

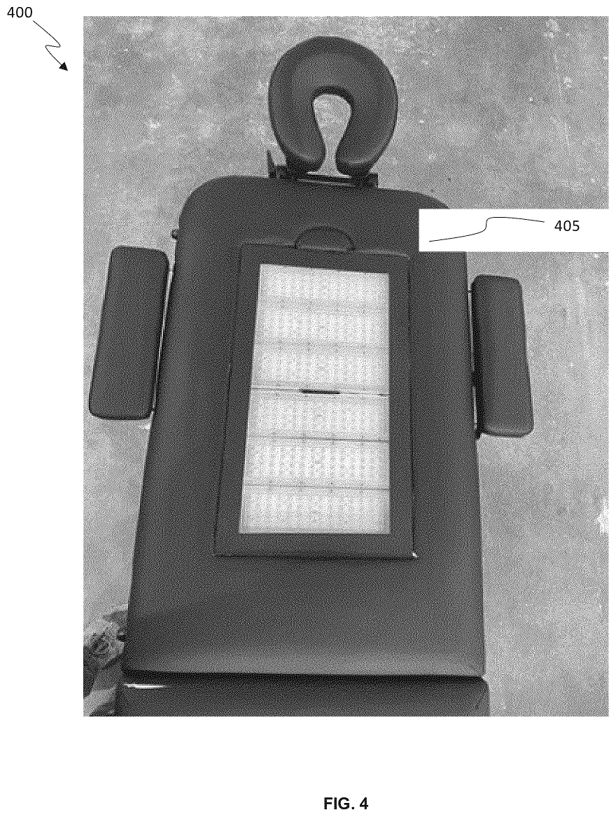

FIG. 4 depicts an exemplary inset high-intensity light source 405 disposed in a removeable component 400 of medical equipment structure, such as a surgical table or examination table, according to embodiments of the present invention. In the example of FIG. 4, high-intensity light source 405 is disposed within a removeable component that can be attached to a table or the like. Typically removable component 405 is attached to other furniture designed to support the patient, such as a base or stand with legs for stability. The removeable component 400 allows light sources of different sizes, types, etc. to be easily installed, and allows components of different sizes and/or strengths to be used to support different body sizes, weights, or to designed for different procedures that require the use of different equipment, for example.

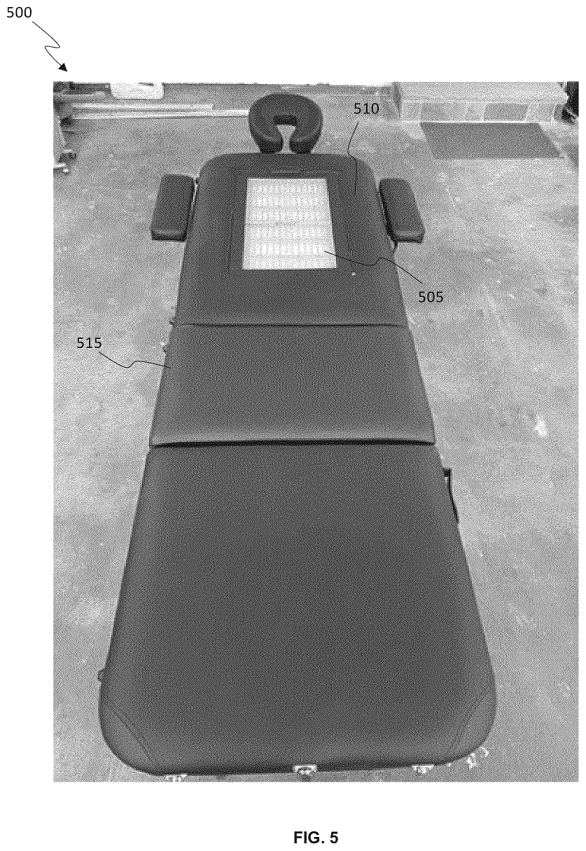

FIG. 5 shows an exemplary inset high-intensity light source 505 disposed in a removeable component 510 attached to medical equipment structure 515 according to embodiments of the present invention. Removeable component 510 can be removed for storage or transportation purposes, and can be attached to the medical equipment structure 515 for use during a medical examination or surgical procedure, for example. The removeable component 510 can advantageously be removed and replaced with another removeable component of a different size, strength, or having a different type of high-intensity light source, for example.

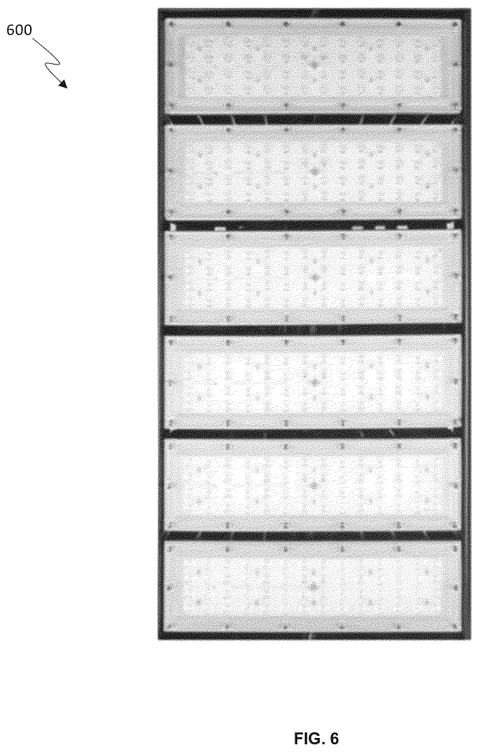

FIG. 6 is a diagram of an exemplary high-intensity light source 600 that can be inset within medical equipment structure to improve the visibility of the practitioner and illuminate aspects of a patient's anatomy (e.g., organs, vasculature, soft-tissue, etc.) by directly illuminating the patient from below according to embodiments of the present invention. In the example of FIG. 6, high-intensity light source 600 is a high-intensity LED array with multiple panels, each panel having 48 individual LEDs. Additional LEDs or fewer LEDs can be used to illuminate the patient, and different types of compact, high-intensity light sources can also be used (e.g., HID, fluorescent, etc.). High-intensity light source 600 can be integrated within a fixed furniture structure, within a cushion or cover of a furniture structure, or within a separate, removeable component thereof. An optional control component can be electrically coupled to high-intensity light source 600 to selectively activate the light, to control the properties of the light, such as the color, brightness, temperature, etc., according to some embodiments.

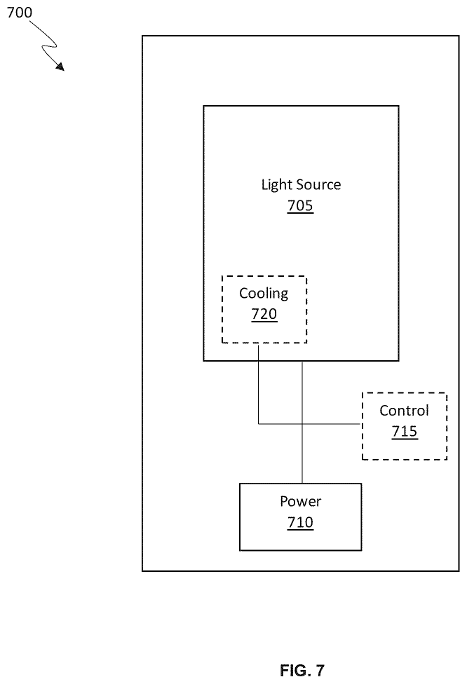

FIG. 7 is a schematic diagram of exemplary medical equipment structure 700 with an inset high-intensity light source 705 for directly illuminating a patient's body from below according to embodiments of the present invention. Light-source 705 can be an LED array, or any other compact high-intensity light source, and is electrically coupled to a power source 710 that drives light-source 705. Power source 710 and/or light source 705 can also be coupled to an optional control element 715 that includes a touch screen or other inputs (e.g., buttons, nobs, etc.) for controlling light-source 705 to produce light with different brightness/intensity, color, temperature, etc. Control element 715 can be integrated into medical equipment structure 700 as a control panel or the like, or can be a separate element that is electrically coupled to the light-source 705 and/or power source 710 by a cable or wire. According to some embodiments, power source 710 is integrated into light source 705, and there can be multiple power sources and light sources integrated into equipment structure 700.

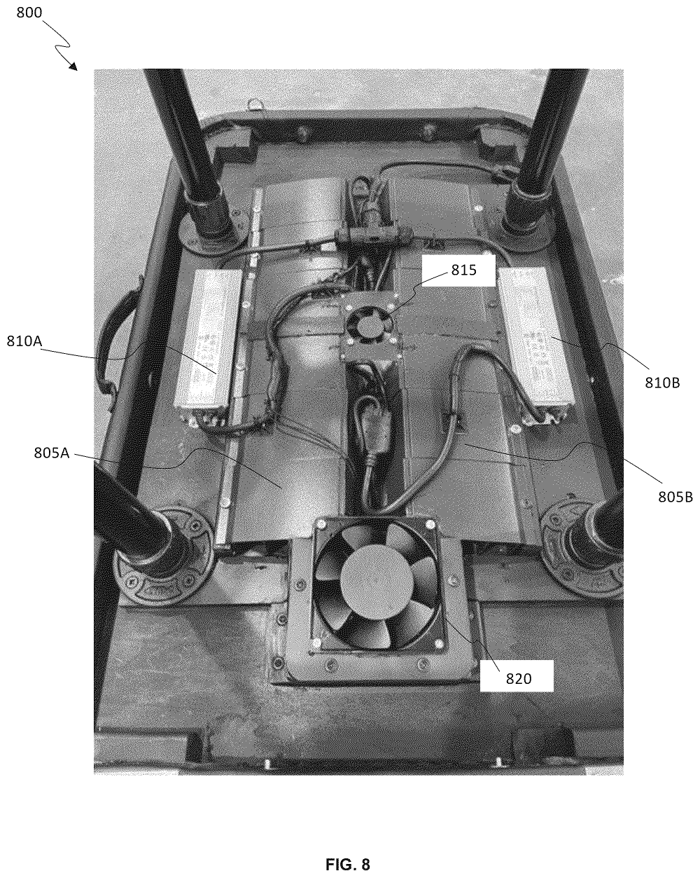

FIG. 8 is a schematic diagram of exemplary medical equipment structure 800 with inset high-intensity light sources light sources 805A and 805B for directly illuminating a patient's body from below and a cooling system including fans 815 and 820 that cool the light sources 805A and 805B according to embodiments of the present invention. Cooling system can include an active fan that is powered externally and blows air over surface of light sources 805A and 805B to facilitate cooling and prevent temperatures from becoming too high. Some embodiments use a liquid-based cooling system that includes a circuit of pipes carrying water or other liquid and a radiator with a fan that exchanges heat from light sources 805A and 805B to facilitate cooling. The liquid can include treated water, glycol or any other liquid suitable for use as a cooling medium.

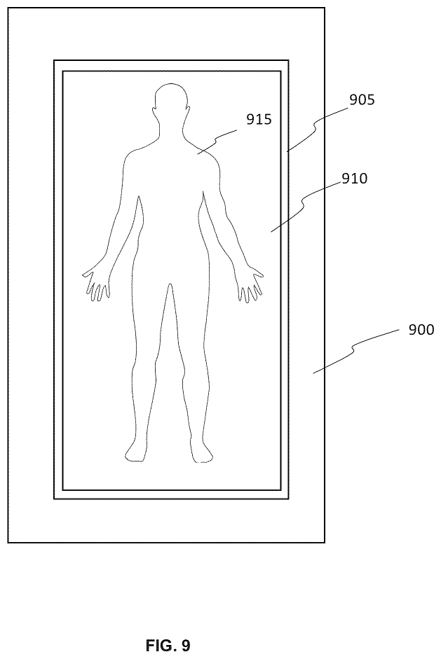

FIG. 9 is a diagram of an exemplary cover, shroud, or aperture 910 for use with a medical equipment structure 900 having inset lighting to prevent light from escaping around the patient's body according to embodiments of the present invention. Cover 910 covers the surface of light source 905 to block and prevent light from leaking from the areas of light source 905 that are not covered by the patient's body. Cover 910 can be created using a 3D printer, for example, based on measurements or images of the patient, or can be made from cloth, fabric, thick paper, or anything that can be created to conform to the shape of the human body (or aspects thereof) and can effectively block light. In the example of FIG. 9, cut-out 915 is shaped to conform to the outline of a patient's entire body. Although FIG. 9 shows a small gap between the edge of light source 905 and cover 910 for clarity, cover 910 typically covers light source 905 from edge to edge with a cut-out potion 915 near the center of cover 910 that allows light to illuminate the patient's body as described above according to embodiments of the present invention.

In addition to the use of a cover or aperture, glasses can be worn by medical professionals during surgery or examination that block certain wavelengths/colors of light to prevent unwanted light emitted from below the patient from becoming visible, or to enhance the visibility of a target region or organ inside the body. The glasses can include polarized lenses, for example.

Embodiments of the present invention are thus described. While the present invention has been described in particular embodiments, it should be appreciated that the present invention should not be construed as limited by such embodiments, but rather construed according to the following claims.

What is claimed is:

1. A medical equipment structure for illuminating a living body, the medical equipment structure comprising:
a flat supportive element; and
a light source disposed within the flat supportive element, wherein the flat supportive element accommodates a living body, and wherein the light source is operable to produce light that illuminates the living body wherein images representing an internal anatomy of the body are visible from outside the living body.

2. The medical equipment structure of claim 1, wherein the flat supportive element comprises a surgical table.

3. The medical equipment structure of claim 1, wherein the flat supportive element comprises an examination table.

4. The medical equipment structure of claim 1, further comprising padding that encloses the flat supportive element, and wherein the light source is partially enclosed by the padding.

5. The medical equipment structure of claim 1, further comprising a control element coupled to the light source, and wherein the control element is operable to adjust at least one of: a brightness; an intensity; a color; and a temperature of the light produced by the light source.

6. The medical equipment structure of claim 1, further comprising a base that supports the flat supportive element, and wherein the flat supportive element is removably coupled to the base.

7. The medical equipment structure of claim 1, wherein the light source comprises an LED array.

8. A surgical table with an integrated light source, the surgical table comprising:
a tabletop comprising an integrated light source; and
a base that supports the tabletop,
wherein the tabletop is operable to accommodate and support a body of a patient, and
wherein the integrated light source is operable to produce light that illuminates the body of the patient from below when the patient is disposed on the tabletop and directly above the integrated light source.

9. The surgical table of claim 8, wherein the tabletop further comprises padding, and wherein the integrated light source is partially enclosed by the padding.

10. The surgical table of claim 8, wherein the integrated light source comprises an LED illumination source.

11. The surgical table of claim 8, wherein the integrated light source comprises an LED array.

12. The surgical table of claim 8, further comprising a control element coupled to the integrated light source, and wherein the control element is operable to adjust at least one of: a brightness; an intensity; a color; and a temperature of the light produced by the integrated light source.

13. The surgical table of claim 12, wherein the integrated light source comprises different types of light sources, and wherein the control element is further operable to selectively activate the different types of light sources.

14. The surgical table of claim 8, wherein the integrated light source is operable to illuminate an internal anatomy of the body of the patient wherein images of the internal anatomy are visible from outside of the body of the patient.

15. A surgical table having a removeable a light source, the surgical table comprising:
a tabletop;
a removable component comprising a light source; and
a base operable to support the tabletop and the removeable component,
wherein the removeable component is removably coupled to at least one of: the base; and the tabletop,
wherein the tabletop accommodations and supports a body of a patient, and
wherein the light source is operable to illuminate the body of the patient from below when the patient is disposed on the tabletop and directly above the light source.

16. The surgical table of claim 15, wherein the removeable component further comprises padding, and wherein the light source is partially enclosed by the padding.

17. The surgical table of claim 15, wherein the light source comprises an LED illumination source.

18. The surgical table of claim 15, wherein the light source comprises an LED array.

19. The surgical table of claim 15, further comprising a control element coupled to the light source, and wherein the control element is operable to adjust at least one of: a brightness; an intensity; a color; and a temperature of the light produced by the light source.

20. The surgical table of claim 19, wherein the light source comprises different types of light sources, and wherein the control element is further operable to selectively activate the different types of light sources.

21. The surgical table of claim 15, wherein the light source is operable to illuminate an internal anatomy of the body of the patient wherein images of internal anatomy are visible from outside of the body of the patient.
